(19) United States
(12) Patent Application Publication
(10) Pub. No.: US 2004/0116510 A1
(43) Pub. Date: Jun. 17, 2004

(54) Antihypertensive agent and cholesterol absorption inhibitor combination therapy

(76) Inventor: Steven A. Nichtberger, Villanova, PA (US)

Correspondence Address: MERCK AND CO INC, P O BOX 2000, RAHWAY, NJ, 070650907

(21) Appl. No.: 10/469,610

(22) PCT Filed: Mar. 5, 2002
(86) PCT No.: PCT/US02/06570

(51) Int. Cl. A61K 31/335

(52) U.S. Cl. 514/449

(57) ABSTRACT

The invention includes methods for treating atherosclerosis and preventing atherosclerotic disease events in a hypertensive patient comprising administering to the patient a therapeutically or prophylactically effective amount of at least one antihypertensive compound in combination with a therapeutically effective amount of a cholesterol absorption inhibitor. The invention also includes a composition comprising at least one antihypertensive compound and a cholesterol absorption inhibitor in therapeutically effective amounts, and a pharmaceutically acceptable carrier.

### FIELD OF THE INVENTION

The instant invention involves a pharmaceutical drug combination comprising at least one antihypertensive compound in combination with a cholesterol absorption inhibitor.

### BACKGROUND OF THE INVENTION

Hypertension is a generally symptomless condition in which abnormally high pressure in the arteries increases the risk of problems such as stroke, aneurysm, heart failure, heart attack, and kidney damage. For an otherwise healthy individual, high blood pressure is defined as a systolic pressure (pressure when the heart contracts) that averages 140 mm Hg or more, a diastolic pressure (pressure when the heart relaxes) that averages 90 mm Hg or more, or both.

The pressure in arteries can be increased in various ways. For one, the heart can pump with more force, putting out more fluid each second. Another possibility is that the large arteries can lose their normal flexibility and become stiff, so that they can't expand when the heart pumps blood through them. Thus, the blood through each heartbeat is forced through less space than normal, and the pressure increases. That is what happens in elderly people whose arterial walls become thickened and stiff because of arteriosclerosis. Blood pressure is similarly increased in vasoconstriction—when the tiny arteries (arterioles) are temporarily constricted as a result of stimulation by nerves or by hormones in the blood. A third way in which the pressure in the arteries can be increased is for more fluid to be added to the system. This happens when the kidneys malfunction and aren't able to remove enough salt and water from the body. The volume of blood in the body increases, so the blood pressure increases.

High blood pressure is treated with a variety of therapeutic agents including thiazide diuretics, adrenergic blockers, angiotensin converting enzyme inhibitors, angiotensin II blockers, calcium antagonists, and direct vasodilators.

Atherosclerosis refers to a disease in which the wall of an artery becomes thicker and less elastic. In atherosclerosis, fatty material accumulates under the inner lining of the arterial wall. Atherosclerosis can affect the arteries of the brain, heart, kidneys, other vital organs, and the arms and legs. When atherosclerosis develops in the arteries that supply the brain (carotid arteries), a stroke may occur; when it develops in the arteries that supply the heart (coronary arteries), a heart attack may occur. Arteries affected with atherosclerosis lose their elasticity, and as the atheromas (patchy thickening in the inner lining of the artery) grow, the arteries narrow. With time, the atheromas may become brittle, and may rupture. Blood may then enter a ruptured atheroma, making it larger, so that it narrows the artery even more. A ruptured atheroma also may spill its fatty contents and trigger the formation of a blood clot (thrombus). The clot may further narrow or even occlude the artery, or it may detach and float downstream where it causes an occlusion (embolism).

Levels of fatty material such as cholesterol and triglycerides may be controlled with a variety of drugs. HMG-CoA reductase inhibitors block the synthesis of cholesterol and enhance the removal of low density lipoproteins from the bloodstream. Fibric acid derivatives lower levels of blood fats, possibly by enhancing fat breakdown. Lipoprotein synthesis inhibitors reduce the rate of very low density lipoprotein production. Bile acid absorbers bind bile acids in the intestine and enhance low density lipoprotein removal from the bloodstream. Renin angiotensin system inhibitors may also decrease the formation of atherosclerotic plaques. Cholesterol absorption inhibitors inhibit intestinal cholesterol absorption.

Clinical studies have demonstrated that members of the HMG-CoA reductase inhibitor class of compounds slow the progression of atherosclerotic lesions in the coronary and carotid arteries. Simvastatin and pravastatin have also been shown to reduce the risk of coronary heart disease events, and in the case of simvastatin a highly significant reduction in the risk of coronary death and total mortality has been shown by the Scandinavian Simvastatin Survival Study. This study also provided some evidence for a reduction in cerebrovascular events. One of the most recent studies, the Heart Protection Study (see www.hpsinfo.org or http://www.ctsu.ox.ac.uk/˜hps/) demonstrated that cholesterol lowering with simvastatin provided life saving benefit for patients with any one risk factor (e.g., hypertension) for cardiovascular events.

The present invention involves the combined administration of at least one antihypertensive compound with a cholesterol absorption inhibitor to prevent atherosclerotic events treat hypertension, atherosclerosis, and related conditions associated with and/or resulting from conditions of hypertension and atherosclerosis. This method is an advantageous alternative means for treating the patient with an antihypertensive compound and a cholesterol lowering drug since, unlike the case with compounds that inhibit cholesterol synthesis, the cholesterol absorption inhibitor does not require titration during treatment, and does not require liver function tests.

### SUMMARY OF THE INVENTION

This invention provides a novel pharmaceutical drug combination therapy comprised of a therapeutically or prophylactically effective amount of at least one antihypertensive compound, particularly one or two antihypertensive compounds, in combination with a therapeutically or prophylactically effective amount of a cholesterol absorption inhibitor.

The drug combination can be used in methods preventing or reducing the risk for occurrence of an atherosclerotic disease event in a hypertensive patient comprising administering a prophylactically effective amount of at least one antihypertensive compound in combination with a prophylactically effective amount of a cholesterol absorption inhibitor to the hypertensive patient.

The drug combination can also be used for treating hypertension, atherosclerosis and/or dyslipidemic conditions such as hypercholesterolemia, elevated serum low density lipoprotein-cholesterol, hypertriglyceridemia and combined hyperlipidemia, comprising administering a therapeutically effective amount of at least one antihypertensive compound, in combination with a therapeutically effective amount of a cholesterol absorption inhibitor to a patient in need thereof. The instant invention encompasses treatment, including preventive treatment, of hypertensive patients who have serum cholesterol levels within the acceptable or “normal” range with respect to total cholesterol, low density lipoprotein (LDL)-cholesterol and high density lipoprotein (HDL)-cholesterol, as well as those having a dyslipidemic condition.

The invention further provides pharmaceutical formulations and compositions and a kit for the drug combination. Additional objects of this invention will become evident from the following detailed description.

### DETAILED DESCRIPTION OF THE INVENTION

This invention provides a method for preventing or reducing the risk for a first or subsequent occurrence of an atherosclerotic disease event in a hypertensive patient comprising administering a prophylactically effective amount of at least one antihypertensive compound in combination with a prophylactically effective amount of a cholesterol absorption inhibitor to the hypertensive patient. The patient may already have atherosclerotic disease at the time of administration, or may be at risk for developing the disease. It also provides a method for preventing or reducing the risk for onset of atherosclerosis in a hypertensive patient comprising administering a prophylactically effective amount of at least one antihypertensive compound in combination with a prophylactically effective amount of a cholesterol absorption inhibitor to the hypertensive patient.

Further provided is a method for treating atherosclerosis in a hypertensive patient comprising administering a therapeutically effective amount of at least one antihypertensive compound in combination with a therapeutically effective amount of a cholesterol absorption inhibitor to a hypertensive patient in need thereof. The invention is also a method for treating atherosclerosis and hypertension in a patient in need thereof comprising administering to the patient a therapeutically effective amount of at least one antihypertensive compound in combination with a therapeutically effective amount of a cholesterol absorption inhibitor.

The invention also encompasses a method for providing lipid management in a hypertensive patient with a dyslipidemic condition selected from the group consisting of hypercholesterolemia, elevated serum LDL-cholesterol level, low serum HDL-cholesterol level, hypertriglyceridemia, combined hyperlipidemia and combinations thereof, comprising administering to the patient a therapeutically effective amount of an antihypertensive compound or compounds in combination with a therapeutically effective amount of a cholesterol absorption inhibitor. Particularly, the invention is also a method for treating hypercholesterolemia, hyperlipidemia, hypertriglyceridemia, lowering LDL-cholesterol levels and/or raising HDL-cholesterol levels in a hypertensive patient comprising administering to the patient a therapeutically effective amount of an antihypertensive compound or compounds in combination with a therapeutically effective amount of a cholesterol absorption inhibitor.

The invention also provides a method for synergistically halting or slowing the progression of atherosclerotic disease once it has become clinically evident in a hypertensive patient with or without hypercholesterolemia comprising administering a therapeutically effective amount of an antihypertensive compound or compounds in combination with a therapeutically effective amount of a cholesterol absorption inhibitor to a patient in need thereof. The invention is also a method for reducing plaque formation by inhibiting inflammation in a hypertensive patient with or without hypercholesterolemia comprising administering a therapeutically effective amount of an antihypertensive compound or compounds in combination with a therapeutically effective amount of a cholesterol absorption inhibitor to a patient in need thereof.

The instant drug combination can also be used for preventing or delaying the progression of renal disease as well as for preventing or reducing the risk for end stage renal failure in a diabetic patient with impaired renal function, particularly a Type II diabetic patient, regardless of the patient's serum cholesterol level or their blood pressure level. For example, the diabetic patients to be treated may be either normotensive or hypertensive. The diabetic patient may also be either normocholesterolemic or hypercholesterolemic, or have a normal or elevated serum LDL-cholesterol level.

The instant drug combination can be used for treatment and/or prevention of one condition or any combination of the conditions described above depending on the medical needs of the individual patient.

In one class of the invention, the antihypertensive compound or compounds are selected from the group consisting of diuretics, beta adrenergic blockers, calcium channel blockers, angiotensin converting enzyme inhibitors, and angiotensin II receptor antagonists. In a subclass of this class, the antihypertensive compound or compounds are selected from a diuretic, an angiotensin II receptor antagonist, and a combination thereof. In a group of this subclass, the angiotensin II receptor antagonist is losartan potassium, and the diuretic is hydrochlorothiazide.

In another class of the invention, the cholesterol absorption inhibitor is a hydroxy-substituted azetidinone cholesterol absorption inhibitor. In a subclass of this class, the hydroxy-substituted azetidinone cholesterol absorption inhibitor is selected from the group consisting of 1-(4fluorophenyl)-3(R)-[3(S)-(4-fluorophenyl)-3-hydroxypropyl)]4(S)-(4-hydroxyphenyl)-2-azetidinone) and 1-(4-fluorophenyl)-3(R)-[3(R)-(4-fluorophenyl)-3-hydroxypropyl)]4(S)-(4-hydroxyphenyl)-2-azetidinone) and the pharmaceutically acceptable salts thereof.

Throughout the specification, the term “at least one antihypertensive compound” includes within its meaning a single antihypertensive compound or two antihypertensive compounds, each of which may be selected from a distinct class of antihypertensives, e.g. an angiotensin II receptor antagonist with a diuretic.

Atherosclerosis encompasses vascular diseases and conditions that are recognized and understood by physicians practicing in the relevant fields of medicine. Atherosclerotic cardiovascular disease, coronary heart disease (also known as coronary artery disease or ischemic heart disease), cerebrovascular disease and peripheral vessel disease are all clinical manifestations of atherosclerosis and are therefore encompassed by the terms “atherosclerosis” and “atherosclerotic disease.” The combination comprised of a therapeutically effective amount of at least one antihypertensive compound in combination with a therapeutically effective amount of a cholesterol absorption inhibitor may be administered to prevent or reduce the risk of occurrence, or recurrence where the potential exists, of a coronary heart disease event, a cerebrovascular event, or intermittent claudication. Coronary heart disease events are intended to include coronary heart disease (CHD) death, fatal and non-fatal myocardial infarction (i.e., a heart attack), and coronary revascularization procedures. Cerebrovascular events are intended to include fatal and non-fatal ischemic or hemorrhagic stroke (also known as cerebrovascular accidents) and transient ischemic attacks. Intermittent claudication is a clinical manifestation of peripheral vessel disease. The term “atherosclerotic disease event” as used herein is intended to encompass coronary heart disease events, cerebrovascular events, and intermittent claudication. It is intended that persons who have previously experienced one or more non-fatal atherosclerotic disease events are those for whom the potential for recurrence of such an event exists.

Persons to be treated with the instant combination therapy include those having hypertension who are at risk for developing atherosclerotic disease or having an atherosclerotic disease event, as well as hypertensive patients who already have atherosclerotic disease and who are at risk for having an atherosclerotic disease event. Included within this group are those having hypertension with or without a lipid disorder such as hyperlipidemia, hypercholesterolemia, hypertriglyceridemia, elevated levels of LDL-cholesterol and low levels of HDL-cholesterol. People identified as having hypertension are those having elevated blood pressure indicated by systolic pressure and diastolic pressure measurements, for example but not limited to blood pressure ≧140/90 mm HG, or those already on antihypertensive medication. Diagnosis of hypertension is well within the purview of the skilled clinician, who will consider all relevant medical factors for the individual patient when making the determination.

Published guidelines for determining those who are at risk for developing atherosclerotic disease or having an atherosclerotic disease event, particularly CHD and CHD events can be found in: the Executive Summary of the Third Report of the National Cholesterol Education Program (NCEP) Expert Panel on Detection, Evaluation, and Treatment of High Blood Cholesterol in Adults (Adult Treatment Panel III), JAMA, 2001; 285 pp.2486-2497, referred to herein as “ATP III,” herein incorporated by reference in its entirety. ATP III also provides lipid treatment guidelines for those who are at risk for or already have established CHD or conditions with an equivalent degree of risk (“risk equivalents”) as CHD. CHD risk equivalents are outlined in ATP III and include diabetes; multiple risk factors that confer a 10 year risk for CHD >20%; and non-CHD clinical forms of atherosclerotic disease such as peripheral arterial disease, abdominal aortic aneurysm and symptomatic carotid artery disease.

For example, as recommended by ATP III, the target LDL-cholesterol level is <100 mg/dl for a patient with CHD or CHD risk equivalents; <130 mg/dl for a patient with 2 or more risk factors; and <160 mg/dl for a patient with 0-1 risk factors. Standard atherosclerotic disease risk factors are known to the average physician practicing in the relevant fields of medicine, and are outlined in ATP III. Such known risk factors include but are not limited to hypertension, cigarette smoking, diabetes, elevated levels of total-cholesterol, elevated levels of LDL-cholesterol (≧200 mg/dl), low levels of HDL-cholesterol (<40 mg/dl), and a family history of premature coronary heart disease.

People who are identified as having one or more of the above-noted risk factors are intended to be included in the group of people considered at risk for developing atherosclerotic disease, particularly CHD. People identified as having one or more of the above-noted risk factors, as well as people who already have atherosclerosis, are intended to be included within the group of people considered to be at risk for having an atherosclerotic disease event.

However, the anti-atherosclerotic benefit of the instant combination drug therapy is not limited by the treatment guidelines described in ATP III. The instant invention also encompasses treatment of a hypertensive patient with the instant combination drug therapy regardless of whether or not the patient has any additional CHD risk factors beyond hypertension, for reducing the risk of onset of atherosclerosis and reducing the risk for occurrence of an atherosclerotic disease event in addition to management of their hypertension. For example, treatment of hypertensive patients with the instant combination drug therapy who are normocholesterolemic and/or have normal serum LDL-cholesterol levels is included within the scope of this invention, as is treatment of hypertensive patients who are hypercholesterolemic and/or have elevated LDL-cholesterol levels.

The term “hypercholesterolemia” and derivatives thereof (i.e., “hypercholesterolemic”) is intended herein to refer to an elevated serum total cholesterol level, i.e., ≧200 mg/dl. The term “normocholesterolemic” is intended herein to refer to a normal serum total cholesterol level, i.e., <200 mg/dl. Whether a given patient has a normal or elevated serum LDL-cholesterol level can be determined by a skilled clinician based on a variety of factors affecting the individual patient including reference to the LDL-cholesterol goals and risk factors described in ATP III. It is important to note that the information presented in ATP III is intended to be used as a guideline, not a strict rule, to assist the clinician with determining what is “normal” and what may warrant treatment with respect to lipid levels for an individual patient. Moreover, the guidelines have evolved over time and will continue to evolve in the future as newer clinical outcomes data becomes available.

Antihypertensive compounds, with dosing information, are identified in the Physician's Desk reference, Edition 53 (1999), and include diuretics, beta adrenergic blockers, calcium channel blockers, angiotensin converting enzyme inhibitors, and angiotensin II receptor antagonists. The term antihypertensive compound includes within its meaning a blood pressure-reducing diuretic, beta adrenergic blocker, calcium channel blocker, angiotensin converting enzyme inhibitor, or angiotensin II receptor antagonist, or a combination of any of these compounds, e.g. a diuretic with an angiotensin II receptor antagonist. Diuretics include, but are not limited to, hydrochlorothiazide, dichlorophenamide, spironolactone, ethacrynic acid, torsemide, furosemide, hydroflumethiazide, and chlorthalidone.

Beta adrenergic blockers include, but are not limited to, sotalol hydrochloride, timolol maleate, carteolol hydrochloride, propranolol hydrochloride, betaxolol hydrochloride, penbutolol sulfate, metoprolol tartrate, metoprolol succinate, acebutolol hydrochloride, and bisoprolol fumarate. Calcium channel blockers include, but are not limited to, nifedipine, verapamil hydrochloride, diltiazem hydrochloride, isradipine, nimodipine, amlodipine besylate, felodipine, nisoldipine, and bepridil hydrochloride.

Angiotensin converting enzyme (ACE) inhibitors include, but are not limited to, quanipril hydrochloride, ramipril, captopril, benazepril hydrochloride, trandolapril, fosinopril sodium, lisinopril, moexipril hydrochloride, and enalapril maleate.

Angiotensin II receptor antagonists include, but are not limited to, candesartan cilexetil ((+/−)-1-(cyclohexylcarbonyloxy)ethyl-2-ethoxy-1-[[2′-(1H-tetrazol-5-yl)biphenyl-4-yl]-1H-benzimidazole-7-carboxylate, U.S. Pat. Nos. 5,703,110 and 5,196,444), eprosartan (3-[1-(4-carboxyphenylmethyl)-2-n-butyl-imidazol-5-yl]-(2-thienylmethyl)-2-propenoic acid, U.S. Pat. Nos. 5,185,351 and 5,650,650), irbesartan 2-n-butyl-3-[[2′-(1H-tetrazol-5-yl)biphenyl4-yl]methyl]1,3-diazazspiro[4,4]non-1-en-4-one, U.S. Pat. Nos. 5,270,317 and 5,352,788), losartan (2-N-butyl4-chloro-5-hydroxymethyl-1-[(2′-(1H-tetrazol-5-yl)biphenyl-4-yl)-methyl]imidazole, potassium salt, U.S. Pat. Nos. 5,138,069, 5,153,197 and 5,128,355), tasosartan (5,8-dihydro-2,4-dimethyl-8-[(2′-(1H-tetrazol-5-yl)[1,1′-biphenyl]4-yl)methyl]-pyrido[2,3-d]pyrimidin-7(6H)-one, U.S. Pat. No. 5,149,699), telmisartan (4′-[(1,4-dimethyl-2′-propyl-(2,6′-bi-1H-benzimidazol)-1′-yl)]-[1,1′-biphenyl]-2-carboxylic acid, U.S. Pat. No. 5,591,762), valsartan ((S)-N-valeryl-N-[[2′-(1H-tetrazol-5-yl)biphenyl-4-yl]methyl]valine, U.S. Pat. No. 5,399,578), and EXP-3137 (2-N-butyl-4-chloro-1-[(2′-(1H-tetrazol-5-yl)biphenyl-4-yl)-methyl]imidazole-5-carboxylic acid, U.S. Pat. Nos. 5,138,069, 5,153,197 and 5,128,355). The contents of these patents are hereby incorporated by reference.

Angiotensin II receptor antagonists also include 3-(2′-(tetrazol-5-yl)-1,1′-biphen-4-yl)methyl-5,7-dimethyl-2-ethyl-3H-imidazo[4,5-b]pyridine, 4′[2-ethyl-4-methyl-6-(5,6,7,8-tetrahydroimidazo[1,2-a]pyridin-2-yl]-benzimidazol-1-yl]-methyl]-1,1′-biphenyl]-2-carboxylic acid, 2-butyl-6-(1-methoxy-1-methylethyl)-2-[2′-(1H-tetrazol-5-yl)biphenyl-4-ylmethyl]guinazolin-4(3H)-one, 3-[(2′-carboxybiphenyl-4-yl)methyl]-2-cyclopropyl-7-methyl-3H-imidazo[4,5-b]pyridine, 2-butyl-4-chloro-1-[(2′-tetrazol-5-yl)biphenyl-4-yl)methyl]imidazole-carboxylic acid, 2-butyl-4-chloro-1-[[2′-(1H-tetrazol-5-yl)[1,1′-biphenyl]4yl]methyl]-1H-imidazole-5-carboxylic acid-1-(ethoxycarbonyl-oxy)ethyl ester potassium salt, dipotassium 2-butyl-4-(methylthio)-1-[[2-[[[(propylamino)carbonyl]amino]-sulfonyl](1,1′-biphenyl)-4-yl]methyl]-1H-imidazole-5-carboxylate, methyl-2-[[4-butyl-2-methyl-6-oxo-5-[[2′-(1H-tetrazol-5-yl)-[1,1′-biphenyl]-4-yl]methyl]-1-(6H)-pyrimidinyl]methyl]-3-thiophencarboxylate, 5-[(3,5-dibutyl-1H-1,2,4-triazol-1-yl)methyl]-2-[2-(1H-tetrazol-5-ylphenyl)]pyridine, 6-butyl-2-(2-phenylethyl)-5-[[2′-(1H-tetrazol-5-yl)[1,1′-biphenyl]-4methyl]pyrimidin (3H)-one D,L lysine salt, 5-methyl-7-n-propyl-8-[[2′-(1H-tetrazol-5-yl)biphenyl4-yl]methyl]-[1,2,4]-triazolo[1,5-c]pyrimidin-2(3H)-one, 2,7-diethyl-5-[[2′-(5-tetrazolyl)biphenyl-4-yl]methyl]-5H-pyrazolo[1,5-b][1,2,4]triazole potassium salt, 2-[2-butyl-4,5-dihydro-4-oxo-3-[2′-(1H-tetrazol-5-yl)-4-biphenylmethyl]-3H-imidazol[4,5-c]pyridine-5-ylmethyl]benzoic acid, ethyl ester, potassium salt, 3-methoxy-2,6-dimethyl-4-[[2′-(1H-tetrazol-5-yl)-1,1′-biphenyl-4-yl]methoxy]pyridine, 2-ethoxy-1-[[2′-(5-oxo-2,5-dihydro-1,2,4-oxadiazol-3-yl)biphenyl-4-yl]methyl]-1H-benzimidazole-7-carboxylic acid, 1-[N-(2′-(1H-tetrazol-5-yl)biphenyl4-yl-methyl)-N-valerolylaminomethyl)cyclopentane-1-carboxylic acid, 7-methyl-2n-propyl-3-[[2′1H-tetrazol-5-yl)biphenyl-4-yl]methyl]-3H-imidazo[4,5-6]pyridine, 2-[5-[(2-ethyl-5,7-dimethyl-3H-imidazo[4,5-b]pyridine-3-yl)methyl]-2-quinolinyl-]sodium benzoate, 2-butyl-6-chloro-4-hydroxymethyl-5-methyl-3-[[2′-(1H-tetrazol-5-yl)biphenyl-4-yl]methyl]pyridine, 2-[[[2-butyl-1-[(4-carboxyphenyl)methyl]-1H-imidazol-5-yl]methyl]amino]benzoic acid tetrazol-5-yl)biphenyl-4-yl]methyl]pyrimidin-6-one, 4(S)-[4-(carboxymethyl)phenoxy]-N-[2(R)-[4-(2-sulfobenzamido)imidazol-1-yl]octanoyl]-L-proline, 1-(2,6-dimethylphenyl)-4-butyl-1,3-dihydro-3-[[6-[2-(1H-tetrazol-5-yl)phenyl]-3-pyridinyl]methyl]-2H-imidazol-2-one, 5,8-ethano-5,8-dimethyl-2-n-propyl-5,6,7,8-tetrahydro-1-[[2′-(1H-tetrazol-5-yl)biphenyl4-yl]methyl]-1H,4H-1,3,4a,8a-tetrazacyclopentanaphthalene-9-one, 4-[1-[2′-(1,2,3,4-tetrazol-5-yl)biphen-4-yl)methylamino]-5,6,7,8-tetrahydro-2-trifylquinazoline, 2-(2-chlorobenzoyl)imino-5-ethyl-3-[2′-(1H-tetrazole-5-yl)biphenyl-4-yl)methyl-1,3,4-thiadiazoline, 2-[5-ethyl-3-[2-(1H-tetrazole-5-yl)biphenyl4-yl]methyl-1,3,4-thiazoline-2-ylidene]aminocarbonyl-1-cyclopentencarboxylic acid dipotassium salt, and 2-butyl4-[N-methyl-N-(3-methylcrotonoyl)amino]-1-[[2′-(1H-tetrazol-5-yl)biphenyl4-yl]methyl]-1H-imidazole-5-carboxylic acid 1-ethoxycarbonyloxyethyl ester.

Angiotensin II receptor antagonists are also described in European patent applications: EP 475,206, EP 497,150, EP 539,086, EP 539,713, EP 535,463, EP 535,465, EP 542,059, EP 497,121, EP 535,420, EP 407,342, EP 415,886, EP 424,317, EP 435,827, EP 433,983, EP 475,898, EP 490,820, EP 528,762, EP 324,377, EP 323,841, EP 420,237, EP 500,297, EP 426,021, EP 480,204, EP 429,257, EP 430,709, EP 434,249, EP 446,062, EP 505,954, EP 524,217, EP 514,197, EP 514,198, EP 514,193, EP 514,192, EP 450,566, EP 468,372, EP 485,929, EP 503,162, EP 533,058, EP 467,207, EP 399,731, EP 399,732, EP 412,848, EP 453,210, EP 456,442, EP 470,794, EP 470,795, EP 495,626, EP 495,627, EP 499,414, EP 499,416, EP 499,415, EP 511,791, EP 516,392, EP 520,723, EP 520,724, EP 539,066, EP 438,869, EP 505,893, EP 530,702, EP 400,835, EP 400,974, EP 401,030, EP 407,102, EP 411,766, EP 409,332, EP 412,594, EP 419,048, EP 480,659, EP 481,614, EP 490,587, EP 467,715, EP 479,479, EP 502,725, EP 503,838, EP 505,098, EP 505,111, EP 513,979, EP 507,594, EP 510,812, EP 511,767, EP 512,675, EP 512,676, EP 512,870, EP 517,357, EP 537,937, EP 534,706, EP 527,534, EP 540,356, EP 461,040, EP 540,039, EP 465,368, EP 498,723, EP 498,722, EP 498,721, EP 515,265, EP 503,785, EP 501,892, EP 519,831, EP 532,410, EP 498,361, EP 432,737, EP 504,888, EP 508,393, EP 508,445, EP 403,159, EP 403,158, EP 425,211, EP 427,463, EP 437,103, EP 481,448, EP 488,532, EP 501,269, EP 500,409, EP 540,400, EP 005,528, EP 028,834, EP 028,833, EP 411,507, EP 425,921, EP 430,300, EP 434,038, EP 442,473, EP 443,568, EP 445,811, EP 459,136, EP 483,683, EP 518,033, EP 520,423, EP 531,876, EP 531,874, EP 392,317, EP 468,470, EP 470,543, EP 502,314, EP 529,253, EP 543,263, EP 540,209, EP 449,699, EP 465,323, EP 521,768, and EP 415,594, which are incorporated by reference into the instant application. Angiotensin II receptor antagonists are also described in PCT patent applications: WO 92/14468, WO 93/08171, WO 93/08169, WO 91/00277, WO 91/00281, WO 91/14367, WO 92/00067, WO 92/00977, WO 92/20342, WO 93/04045, WO 93/04046, WO 91/15206, WO 92/14714, WO 92/09600, WO 92/16552, WO 93/05025, WO 93/03018, WO 91/07404, WO 92/02508, WO 92/13853, WO 91/19697, WO 91/11909, WO 91/12001, WO 91/11999, WO 91/15209, WO 91/15479, WO 92/20687, WO 92/20662, WO 92/20661, WO 93/01177, WO 91/17771, WO 91/14679, WO 91/13063, WO 92/13564, WO 91/17148, WO 91/18888, WO 91/19715, WO 92/02257, WO 92/04335, WO 92/05161, WO 92/07852, WO 92/15577, WO 93/03033, WO 91/16313, WO 92/00068, WO 92/02510, WO 92/09278, WO 9210179, WO 92/10180, WO 92/10186, WO 92/10181, WO 92/10097, WO 92/10183, WO 92/10182, WO 92/10187, WO 92/10184, WO 92/10188, WO 92/10180, WO 92/10185, WO 92/20651, WO 93/03722,WO 93/06828,WO 93/03040, WO 92/19211, WO 92/22533, WO 92/06081, WO 92/05784,WO 93/00341, WO 92/04343, WO 92/04059, and WO 92/05044, which are incorporated by reference into the instant application.

Angiotensin II receptor antagonists are also described in U.S. patents: U.S. Pat. No. 5,104,877, U.S. Pat. No. 5,187,168, U.S. Pat. No. 5,149,699, U.S. Pat. No. 5,185,340, U.S. Pat. No. 4,880,804, U.S. Pat. No. 5,138069, U.S. Pat. No. 4,916,129, U.S. Pat. No. 5,153,197, U.S. Pat. No. 5,173,494, U.S. Pat. No. 5,137,906, U.S. Pat. No. 5,155,126, U.S. Pat. No. 5,140,037, U.S. Pat. No. 5,137,902, U.S. Pat. No. 5,157,026, U.S. Pat. No. 5,053,329, U.S. Pat. No. 5,132,216, U.S. Pat. No. 5,057,522, U.S. Pat. No. 5,066,586, U.S. Pat. No. 5,089,626, U.S. Pat. No. 5,049,565, U.S. Pat. No. 5,087,702, U.S. Pat. No. 5,124,335, U.S. Pat. No. 5,102,880, U.S. Pat. No. 5,128,327, U.S. Pat. No. 5,151,435, U.S. Pat. No. 5,202,322, U.S. Pat. No. 5,187,159, U.S. Pat. No. 5,198,438, U.S. Pat. No. 5,182,288, U.S. Pat. No. 5,036,048, U.S. Pat. No. 5,140,036, U.S. Pat. No. 5,087,634, U.S. Pat. No. 5,196,537, U.S. Pat. No. 5,153,347, U.S. Pat. No. 5,191,086, U.S. Pat. No. 5,190,942, U.S. Pat. No. 5,177,097, U.S. Pat. No. 5,212,177, U.S. Pat. No. 5,208,234, U.S. Pat. No. 5,208,235, U.S. Pat. No. 5,212,195, U.S. Pat. No. 5,130,439, U.S. Pat. No. 5,045,540, U.S. Pat. No. 5,041,152, and U.S. Pat. No. 5,210,204, which are incorporated by reference into the instant application.

Cholesterol absorption inhibitors block the movement of cholesterol from the intestinal lumen into enterocytes of the small intestinal wall. This blockade is their primary mode of action in reducing serum cholesterol levels. These compounds are distinct from compounds which reduce serum cholesterol levels primarily by mechanisms of action such as acyl coenzyme A—cholesterol acyl transferase (ACAT) inhibition, inhibition of triglyceride synthesis, MIP inhibition, bile acid sequestration, and transcription modulation such as agonists or antagonists of nuclear hormones.

Cholesterol absorption inhibitors are described in U.S. Pat. No. 5,846,966, U.S. Pat. No. 5,631,365, U.S. Pat. No. 5,767,115, U.S. Pat. No. 6,133,001, U.S. Pat. No. 5,886,171, U.S. Pat. No. 5,856,473, U.S. Pat. No. 5,756,470, U.S. Pat. No. 5,739,321, U.S. Pat. No. 5,919,672, WO 00/63703, WO /0060107, WO 00/38725, WO 00/34240, WO 00/20623, WO 97/45406, WO 97/16424, WO 97/16455, and WO 95/08532, the entire contents of which are hereby incorporated by reference. An examplary cholesterol absorption inhibitor is ezetimibe, which is 1-(4-fluorophenyl)-3(R)-[3(S)-hydroxy-3-(4-fluorophenyl)propyl]4(S)-(4-hydroxyphenyl)-2-azetidinone, shown below as

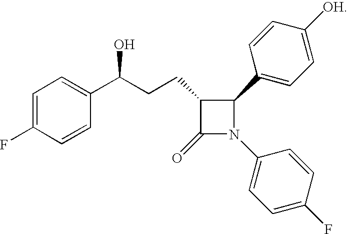

Additional examplary hydroxy-substituted azetidinone cholesterol absorption inhibitors are specifically described in U.S. Pat. No. 5,767,115, column 39, lines 54-61 and column 40, lines 1-51 (hereby incorporated by reference), represented by the formula

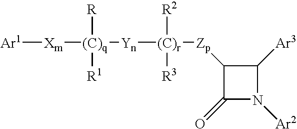

as defined in column 2, lines 20-63 (hereby incorporated by reference).

These and other cholesterol absorption inhibitors within the scope of the present invention can be identified according to the assay of hypolipidemic compounds using the hyperlipidemic hamster described in U.S. Pat. No. 5,767,115, column 19, lines 47-65 (hereby incorporated by reference), in which hamsters are fed a controlled cholesterol diet and dosed with test compounds for seven days. Plasma lipid analysis is conducted and data is reported as percent reduction of lipid versus control.

The compounds of use in this invention may have one or more chiral centers and the present compounds may occur as racemates, racemic mixtures and as individual diasteriomers or enantiomers with all such isomeric forms and mixtures thereof being included within the scope of this invention. Furthermore, some of the crystalline forms for compounds of the present invention may exist as polymorphs and as such are intended to be included in the present invention. In addition, some of the compounds of the instant invention may form solvates with water or common organic solvents. Such solvates and hydrates, as well as anhydrous compositions, are encompassed within the scope of this invention. Some of the compounds described herein may contain olefinic double bonds, and unless specified otherwise, are meant to include both E and Z geometric isomers.

Herein, the term “pharmaceutically acceptable salts” shall mean non-toxic salts of the compounds employed in this invention which are generally prepared by reacting the free acid with a suitable organic or inorganic base, particularly those formed from cations such as sodium, potassium, aluminum, gold, silver, calcium, lithium, magnesium, zinc and tetramethylammonium, as well as those salts formed from amines such as ammonia, alkyl amines, hydroxy alkyl amines, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenzimidazole, diethylamine, piperazine, morpholine, 2,4,4-trimethyl-2-pentamine and tris(hydroxymethyl)-aminomethane. Suitable acids for salt formation include hydrochloric, sulfuric, phosphoric, acetic, citric, oxalic, malonic, salicylic, malic, fumaric, succinic, ascorbic, maleic, methanesulfonic, and other mineral and carboxylic acids known tothose skilled in the art. Pharmaceutically acceptable esters include, but are not limited to, —C₁₋₄ alkyl esters and —C₁₋₄ alkyl esters substituted with phenyl-, dimethylamino-, and acetylamino. “C₁₋₄ alkyl” herein includes straight or branched aliphatic chains containing from 1 to 4 carbon atoms, for example methyl, ethyl, n-propyl, n-butyl, iso-propyl, sec-butyl and tert-butyl. Ester derivatives of the described compounds may act as prodrugs which, when absorbed into the bloodstream of a warm-blooded animal, may cleave in such a manner as to release the drug form and permit the drug to afford improved therapeutic efficacy.

The instant pharmaceutical drug combination comprising at least one antihypertensive compound in combination with a cholesterol absorption inhibitor includes a single pharmaceutical dosage formulation which contains both the antihypertensive compound and the cholesterol absorption inhibitor, as well as administration of each active compound in its own separate pharmaceutical dosage formulation. Where separate dosage formulations are used, the antihypertensive compound and the cholesterol absorption inhibitor can be administered at essentially the same time, i.e., concurrently, or at separately staggered times, i.e., sequentially. The instant pharmaceutical drug combination is understood to include all these regimens. Administration in these various ways are suitable for the present invention as long as the beneficial pharmaceutical effect of the antihypertensive compound and the cholesterol absorption inhibitor are realized by the patient at substantially the same time. Such beneficial effect is preferably achieved when the target blood level concentrations of each active drug are maintained at substantially the same time. It is preferred that the antihypertensive compound and the cholesterol absorption inhibitor be co-administered concurrently on a once-a-day dosing schedule; however, varying dosing schedules, such as the antihypertensive compound once, twice or more times per day and the cholesterol absorption inhibitor once, twice or more times per day, is also encompassed herein. A single oral dosage formulation comprised of both the antihypertensive compound and the cholesterol absorption inhibitor is preferred. A single dosage formulation will provide convenience for the patient, which is an important consideration especially for patients who already have coronary heart disease and may be in need of multiple medications.

The term “patient” includes mammals, especially humans, who take an antihypertensive compound or compounds in combination with a cholesterol absorption inhibitor for any of the uses described herein. Administering of the drug combination to the patient includes both self-administration and administration to the patient by another person.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician

The dosage regimen utilizing at least one antihypertensive compound in combination with a cholesterol absorption inhibitor is selected in accordance with a variety of factors including type, species, age, weight, sex and medical condition of the patient; the severity of the condition to be treated; the route of administration; the renal and hepatic function of the patient; and the particular compound or salt or ester thereof employed. Since two different active compounds are being used together in a combination therapy, the potency of each of the compounds and the interactive effects achieved by combining them together must also be taken into account. A consideration of these factors is well within the purview of the ordinarily skilled clinician for the purpose of determining the therapeutically effective or prophylactically effective dosage amounts needed to prevent, counter, or arrest the progress of the condition.

An effective amount of the antihypertensive compound or compounds for use in the methods of this invention include daily dosage amounts between about 0.001 mg and 2000 mg, e.g. 0.1, 1.0, 2.5, 5, 10, 12.5, 20, 25, 30, 40, 50, 60, 80, 100, 120, 200, 300, 360, 400, 480 or 600 mg of the active ingredient in single or divided doses, or in sustained release form. As an example, the daily dosage amount of losartan potassium can be 25, 50 or 100 mg. As a further example, the daily dosage amounts for losartan with hydrochlorothiazide can be 50 mg losartan with 12.5 mg hydrochlorothiazide; or 100 mg losartan with 25 mg hydrochlorothiazide.

An effective amount of cholesterol absorption inhibitor for use in the methods of this invention include dosages of from about 0.1 to about 30 mg/kg of body weight per day, preferably about 0.1 to about 15 mg/kg. For an average body weight of 70 kg, the dosage level is therefore from about 7 mg to about 2100 mg of drug per day, e.g. 10, 20, 40, 100 and 200 mg per day, in single or divided doses, or in sustained release form. As a further example, the daily dosage amounts for ezetimibe 10 mg. This dosage regimen may be adjusted to provide the optimal therapeutic response.

For example, the instant pharmaceutical drug combination administered in a single daily dose may be comprised of about 0.001 mg to about 2000 mg antihypertensive compound in combination with about 7 mg to about 2100 mg of the cholesterol absorption inhibitor. As a particular example, the medicament may be comprised of 100 mg antihypertensive compound in combination with 100 mg of cholesterol absorption inhibitor.

Dosage amounts for each active drug will vary depending on factors as noted above, including the potency of the particular compound. Although the combination drug therapy of the present invention may be administered in divided doses, for example from one to four times daily, a single daily dose of each active drug is preferred, and most preferably the antihypertensive compound or compounds and the cholesterol absorption inhibitor are combined in a once-daily oral dosage unit.

The instant combination therapy can be administered chronically in order to control the patient's blood pressure and cholesterol and triglyceride levels, and in order to gain the long-term benefits of hypertension and atherosclerotic disease treatment and prevention; the drug combination can also be administered acutely when warranted.

Additional active agents may be used in combination with the antihypertensive compound and the cholesterol absorption inhibitor in a single dosage formulation, or may be administered to the patient in a separate dosage formulation, which allows for concurrent or sequential administration. One or more additional active agents may be administered with the instant combination therapy. Examples of additional active agents which may be employed include but are not limited to HMG-CoA synthase inhibitors; squalene epoxidase inhibitors; squalene synthetase inhibitors (also known as squalene synthase inhibitors), acyl-coenzyme A: cholesterol acyltransferase (ACAT) inhibitors including selective inhibitors of ACAT-1 or ACAT-2 as well as dual inhibitors of ACAT-1 and -2; microsomal triglyceride transfer protein (MTP) inhibitors; probucol; niacin; bile acid sequestrants; LDL (low density lipoprotein) receptor inducers; platelet aggregation inhibitors, for example glycoprotein IIb/IIIa fibrinogen receptor antagonists and aspirin; human peroxisome proliferator activated receptor gamma (PPARγ) agonists including the compounds commonly referred to as glitazones for example troglitazone, pioglitazone and rosiglitazone and, including those compounds included within the structural class known as thiazolidinediones as well as those PPARγ agonists outside the thiazolidinedione structural class; PPARα agonists such as clofibrate, fenofibrate including micronized fenofibrate, and gemfibrozil; PPAR dual α/γ agonists; vitamin B₆ (also known as pyridoxine) and the pharmaceutically acceptable salts thereof such as the HCl salt; vitamin B₁₂ (also known as cyanocobalamin); folic acid or a pharmaceutically acceptable salt or ester thereof such as the sodium salt and the methylglucamine salt; anti-oxidant vitamins such as vitamin C and E and beta carotene; endothelian antagonists; agents that enhance ABC1 gene expression; FXR and LXR ligands including both inhibitors and agonists; bisphosphonate compounds such as alendronate sodium; and cyclooxygenase-2 inhibitors such as rofecoxib and celecoxib.

The invention is also a pharmaceutical composition comprising at least one antihypertensive compound, particularly one or two antihypertensive compounds, and a cholesterol absorption inhibitor in therapeutically effective amounts, and a pharmaceutically acceptable carrier. In one class of the composition, the antihypertensive compound or compounds are selected from losartan potassium, hydrochlorothiazide, and a combination thereof. In a subclass of the composition, the cholesterol absorption inhibitor is selected from the group consisting of 1-(4-fluorophenyl)-3(R)-[3(S)-(4-fluorophenyl)-3-hydroxypropyl]-4(S)-(4-hydroxyphenyl)-2-azetidinone and 1-(4-fluorophenyl)-3(R)-[3(R)-(4-fluorophenyl)-3-hydroxypropyl]-4(S)-(4-hydroxyphenyl)-2-azetidinone and the pharmaceutically acceptable salts and esters thereof. Preferably, the cholesterol absorption inhibitor is ezetimibe.

The active compounds employed in the instant combination therapy can be administered in such oral forms as tablets, capsules, pills, powders, granules, elixirs, tinctures, suspensions, syrups, and emulsions. The instant invention includes the use of both oral rapid-release and time-controlled release pharmaceutical formulations, as well as enteric coated formulations. A particular example of an oral time-controlled release pharmaceutical formulation is described in U.S Pat. No. 5,366,738. Oral formulations are preferred. Such pharmaceutical compositions are known to those of ordinary skill in the pharmaceutical arts; for example, see Remington's Pharmaceutical Sciences, Mack Publishing Co., Easton, Pa.

In the methods of the present invention, the active agents are typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with a non-toxic, pharmaceutically acceptable, inert carrier such as lactose, starch, sucrose, glucose, modified sugars, modified starches, methyl cellulose and its derivatives, dicalcium phosphate, calcium sulfate, mannitol, sorbitol and other reducing and non-reducing sugars, magnesium stearate, steric acid, sodium stearyl fumarate, glyceryl behenate, calcium stearate and the like. For oral administration in liquid form, the drug components can be combined with non-toxic, pharmaceutically acceptable inert carrier such as ethanol, glycerol, water and the like. Moreover, when desired or necessary, suitable binders, lubricants, disintegrating agents and coloring and flavoring agents can also be incorporated into the mixture. Stabilizing agents such as antioxidants (BHA, BHT, propyl gallate, sodium ascorbate, citric acid) can also be added to stabilize the dosage forms. Other suitable components include gelatin, sweeteners, natural and synthetic gums such as acacia, tragacanth or alginates, carboxymethylcellulose, polyethylene glycol, waxes and the like.

The active drugs can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Active drug may also be delivered by the use of monoclonal antibodies as individual carriers to which the compound molecules are coupled. Active drug may also be coupled with soluble polymers as targetable drug carriers. Such polymers can include polyvinyl-pyrrolidone, pyran copolymer, polyhydroxy-propyl-methacrylamide-phenol, polyhydroxy-ethyl-aspartamide-phenol, or polyethyleneoxide-polylysine substituted with palmitoyl residues. Furthermore, active drug may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Although the active agents of the present method may be administered in divided doses, for example two or three times daily, a single daily dose of each of the antihypertensive compounds and the cholesterol absorption inhibitors is preferred, with a single daily dose of both agents in a single pharmaceutical composition being most preferred.

The instant invention also encompasses a process for preparing a pharmaceutical composition comprising combining therapeutically or prophylactically effective amounts of each of the antihypertensive compound or compounds and the cholesterol absorption inhibitor with a pharmaceutically acceptable carrier, as well as the pharmaceutical composition which is made by combining therapeutically or prophylactically effective amounts of each of the antihypertensive compound or compounds and the cholesterol absorption inhibitor with a pharmaceutically acceptable carrier.

This invention further encompasses a kit comprising at least one antihypertensive compound in one container and a cholesterol absorption inhibitor in a separate container, each in therapeutically effective amounts with a pharmaceutically acceptable carrier. The term “kit” as used herein encompasses packaging designed to provide the patient with one or more days worth of the drug combination therapy, wherein the antihypertensive component and the cholesterol absorption inhibitor component of the drug combination are each contained in their own separate oral dosage units, preferably each in its own once-daily oral dosage unit. Therefore, there may be one or multiple dosage units for each of the antihypertensive agent and the cholesterol absorption inhibitor in the kit, depending on the number of days of treatment intended to be provided by the kit. A dosage unit is intended to mean a pharmaceutical dosage formulation that delivers a discrete quantity of drug to the patient, such as a tablet, capsule or the like.

Particularly, the kit is comprised of a therapeutically effective amount of at least one antihypertensive compound in one or more oral dosage units and a therapeutically effective amount of a cholesterol absorption inhibitor in one or more separate oral dosage units, such that there are an equal number of antihypertensive dosage units and cholesterol absorption inhibitor dosage units in the kit. The same antihypertensive compound or combination of compounds would be used for all the antihypertensive dosage units in any one kit, and similarly the same cholesterol absorption inhibitor would be used for all the cholesterol absorption inhibitor dosage units in any one kit.

In one class of the kit, the antihypertensive oral dosage unit is comprised of a compound or compounds selected from losartan potassium, hydrochlorothiazide, and a combination thereof. In a second class of the kit, the cholesterol absorption inhibitor oral dosage unit is comprised of a compound selected from the group consisting of 1-(4-fluorophenyl)-3(R)-[3(S)-(4-fluorophenyl)-3-hydroxypropyl]-4(S)-(4-hydroxyphenyl)-2-azetidinone and 1-(4-fluorophenyl)-3(R)-[3(R)-(4-fluorophenyl)-3-hydroxypropyl]-4(S)-(4-hydroxyphenyl)-2-azetidinone and the pharmaceutically acceptable salts and esters thereof. In a subclass of the kit, the antihypertensive oral dosage unit is comprised of a compound or compounds selected from losartan potassium, hydrochlorothiazide, and a combination thereof, and the cholesterol absorption inhibitor oral dosage unit is comprised of ezetimibe.

The packaging for the kit could be designed and manufactured in a variety of ways. One example includes but is not limited to a blister package containing rows of a losartan tablet or a losartan/hydrochlorothiazide tablet and an ezetimibe tablet placed side by side on the same blister card, each of the two tablets in its own blister bubble, with calendar or similar type markings on the card that convey to the user that one “pair” of tablets (i.e., one antihypertensive tablet and one cholesterol absorption inhibitor tablet) is to be ingested per day.

Therapeutically effective amounts of antihypertensive compound or compounds and cholesterol absorption inhibitor can be used together for the preparation of a medicament useful for treating or preventing any of the medical conditions described herein, in dosage amounts described herein. For example, the medicament may be useful for treating hypertension, preventing or reducing the risk of developing atherosclerotic disease, halting or slowing the progression of atherosclerotic disease once it has become clinically manifest, and preventing or reducing the risk of a first or subsequent occurrence of an atherosclerotic disease event.

The instant invention also encompasses the use of a therapeutically effective amount of an antihypertensive compound or compounds for the preparation of a medicament for combined use with a therapeutically effective amount of a cholesterol absorption inhibitor for treating hypertension, and/or for halting or slowing the progression of atherosclerotic disease. It also encompasses the use of a therapeutically effective amount of an antihypertensive compound or compounds for the preparation of a medicament for the combined use with a prophylactically effective amount of a cholesterol absorption inhibitor for preventing or reducing the risk of developing atherosclerotic disease, or for preventing or reducing the risk of occurrence or recurrence of an atherosclerotic disease event, or for delaying the progression of renal disease as described herein. The medicament or pharmaceutical drug combination comprised of the antihypertensive compound or compounds and the cholesterol absorption inhibitor may also be prepared with one or more additional active agents, such as those described above.

### EXAMPLE 1

A patient having high blood pressure is administered 100 mg per day of losartan potassium. The patient also receives 10 mg of ezetimibe each day.

### EXAMPLE 2

A patient having high blood pressure is administered 50 mg per day of losartan potassium. The patient also receives 10 mg of ezetimibe each day.

### EXAMPLE 3

A patient having high blood pressure is administered a tablet comprising 50 mg of losartan potassium and 12.5 mg of hydrochlorothiazide twice each day. The patient also receives 10 mg of ezetimibe each day.

### EXAMPLE 4

A patient having high blood pressure is administered 10 mg per day of enalapril maleate. The patient also receives 10 mg of ezetimibe each day.

### EXAMPLE 5

A patient having high blood pressure is administered 20 mg per day of enalapril maleate. The patient also receives 10 mg of ezetirnibe each day.

### EXAMPLE 6

A patient having high blood pressure is administered 60 mg per day of timolol maleate. The patient also receives 10 mg of ezetimibe each day.

### EXAMPLE 7

A patient having high blood pressure and elevated total cholesterol levels (greater than 200 mg/dL) is administered an antihypertensive compound and a cholesterol absorption inhibitor according to one of the treatments described in Examples 1-6.

### EXAMPLE 8

A patient having normal blood pressure and elevated total cholesterol levels (greater than 200 mg/dL) is administered an antihypertensive compound and a cholesterol absorption inhibitor according to one of the treatments described in Examples 1-6.

### EXAMPLE 9

A patient having high blood pressure and normal total cholesterol levels (less than or equal to 200 mg/dL) is administered an antihypertensive compound and a cholesterol absorption inhibitor according to one of the treatments described in Examples 1-6.

### EXAMPLE 10

A patient having high blood pressure and normal total cholesterol levels (less than or equal to 200 mg/dL), is administered an antihypertensive compound and a cholesterol absorption inhibitor according to one of the treatments described in Examples 1-6, wherein the patient has none of the following risk factors: diabetes, atherosclerosis, cigarette smoking, elevated LDL-cholesterol level, low serum HDL-cholesterol level, family history of premature coronary heart disease.

### EXAMPLE 11

A patient having high blood pressure and normal total cholesterol levels (less than or equal to 200 mg/dL), is administered an antihypertensive compound and a cholesterol absorption inhibitor according to one of the treatments described in Examples 1-6, wherein the patient has none of the following risk factors: diabetes, atherosclerosis, cigarette smoking, elevated LDL-cholesterol level, low serum HDL-cholesterol level, family history of premature coronary heart disease, being male ≧45 years of age, and being female ≧55 years of age.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the patient being treated for any of the indications for the active agents used in the instant invention as indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A method for preventing or reducing the risk for occurrence of an atherosclerotic disease event in a hypertensive patient comprising administering a prophylactically effective amount of at least one antihypertensive compound in combination with a prophylactically effective amount of a cholesterol absorption inhibitor to the hypertensive patient.

2. The method of claim 1 wherein the patient is hypercholesterolemic.

3. The method of claim 1 wherein the patient is normocholesterolemic.

4. The method of claim 1 wherein the patient has an elevated serum LDL-cholesterol level.

5. The method of claim 1 wherein the patient has a normal serum LDL-cholesterol level.

6. The method of claim 1 wherein the hypertensive patient is not diabetic and does not have atherosclerotic disease.

7. The method of claim 6 wherein the patient does not have any risk factors in the group consisting of: cigarette smoking; low serum HDL-cholesterol level; family history of premature coronary heart disease; being male ≧45 years of age; and being female ≧55 years of age.

8. The method of claim 7 wherein the patient has a fasting serum LDL-cholesterol level <160 mg/dl.

9. The method of claim 7 wherein the patient has a fasting serum L)L-cholesterol level >160 mg/dl.

10. The method of claim 6 wherein the patient has one or more risk factors in addition to hypertension selected from the group consisting of: cigarette smoking; low serum HDL-cholesterol level, family history of premature coronary heart disease, being male ≧45 years of age, and being female ≧55 years of age.

11. The method of claim 10 wherein the patient has a fasting serum LDL-cholesterol level <130 mg/dl.

12. The method of claim 10 wherein the patient has a fasting serum LDL-cholesterol level ≧130 mg/dl.

13. The method of claim 1 wherein the patient has at least one condition selected from the group consisting of diabetes and atherosclerotic disease.

14. The method of claim 13 wherein the patient has a fasting serum LDL-cholesterol level <100 mg/dl.

15. The method of claim 13 wherein the patient has a fasting serum LDL-cholesterol level ≧100 mg/dl.

16. The method of claim 1 wherein the atherosclerotic disease event is selected from the group consisting of: a coronary heart disease event, a cerebrovascular event, and intermittent claudication.

17. The method of claim 16 wherein the atherosclerotic disease event is selected from coronary heart disease death, myocardial infarction, and stroke.

18. The method of claim 1 wherein the at least one antihypertensive compound is selected from the group consisting of diuretics, beta adrenergic blockers, calcium channel blockers, angiotensin converting enzyme inhibitors, and angiotensin II receptor antagonists.

19. The method of claim 18 wherein the at least one antihypertensive compound is selected from the group consisting of an angiotensin II receptor antagonist, a diuretic and a combination thereof.

20. The method of claim 19, wherein the angiotensin II receptor antagonist is losartan potassium and the diuretic is hydrochlorothiazide.

21. The method of claim 1 wherein the cholesterol absorption inhibitor is a hydroxy-substituted azetidinone cholesterol absorption inhibitor.

22. The method of claim 21 wherein the hydroxy-substituted azetidinone cholesterol absorption inhibitor is ezetimibe.

23. The method of claim 22 wherein the at least one antihypertensive compound is selected from the group consisting of (a) losartan potassium, (b) hydrochlorothiazide and (c) losartan potassium and hydrochlorothiazide.

24. A method for preventing or reducing the risk for onset of atherosclerosis in a hypertensive patient comprising administering a prophylactically effective amount of at least one antihypertensive compound in combination with a prophylactically effective amount of a cholesterol absorption inhibitor to the hypertensive patient.

25. A method for treating atherosclerosis in a hypertensive patient comprising administering a therapeutically effective amount of at least one antihypertensive compound in combination with a therapeutically effective amount of a cholesterol absorption inhibitor to a hypertensive patient in need thereof.

26. A method for treating atherosclerosis and hypertension comprising administering a therapeutically effective amount of at least one antihypertensive compound in combination with a therapeutically effective amount of a cholesterol absorption inhibitor to a patient in need thereof.

27. A method for providing lipid management in a hypertensive patient comprising administering a therapeutically effective amount of at least one antihypertensive compound in combination with a therapeutically effective amount of a cholesterol absorption inhibitor to a patient in need thereof.

28. The method of claim 27 wherein the patient has a dyslipidemic condition selected from the group consisting of hypercholesterolemia, elevated serum LDL-cholesterol level, hypertriglyceridemia, combined hyperlipidemia and low HDL-cholesterol level.

29. The method of claim 28 wherein the patient has atherosclerosis.

30. A method for reducing atherosclerotic progression in a hypertensive patient comprising administering a therapeutically effective amount of at least one antihypertensive compound in combination with a therapeutically effective amount of a cholesterol absorption inhibitor to a patient in need thereof.

31. A method for reducing plaque formation in a hypertensive patient comprising administering a therapeutically effective amount of at least one antihypertensive compound in combination with a therapeutically effective amount of a cholesterol absorption inhibitor to a patient in need thereof.

32. A method for preventing or reducing the risk for end stage renal failure in a diabetic patient with impaired renal function comprising administering a therapeutically effective amount of at least one antihypertensive compound in combination with a therapeutically effective amount of a cholesterol absorption inhibitor to a patient in need thereof.

33. The method of claim 32 wherein the patient has Type II diabetes.

34. A pharmaceutical composition comprising at least one antihypertensive compound and a cholesterol absorption inhibitor in therapeutically effective amounts, and a pharmaceutically acceptable carrier.

35. The composition of claim 34 wherein the at least one antihypertensive compound is selected from the group consisting of (a) losartan potassium, (b) hydrochlorothiazide, and (c) losartan potassium and hydrochlorothiazide.

36. The composition of claim 34 wherein the cholesterol absorption inhibitor is ezetimibe.

37. A process for preparing the pharmaceutical composition of claim 22 comprising combining the antihypertensive compound or compounds with the cholesterol absorption inhibitor and the pharmaceutically acceptable carrier.

38. A pharmaceutical composition made by combining at least one antihypertensive compound and a cholesterol absorption inhibitor in therapeutically effective amounts, and a pharmaceutically acceptable carrier.

39. A kit comprising a therapeutically effective amount of at least one antihypertensive compound in at least one oral dosage unit and a therapeutically effective amount of a cholesterol absorption inhibitor in at least one separate oral dosage unit.
